(19) United States
(12) Patent Application Publication
(10) Pub. No.: US 2005/0132461 A1
(43) Pub. Date: Jun. 23, 2005

(54) Hat with shade panel

(75) Inventor: Dong Keun Koo, Fort Lee, NJ (US)

Correspondence Address: John K. Kim;McCarter & English, LLP, Four Gateway Center, 100 Mulberry Street, Newark, NJ, 07102, US

(73) Assignee: V3 Import Corporation

(21) Appl. No.: 10/848,284

(22) Filed: May 18, 2004

(30) Foreign Application Priority Data
Dec. 19, 2003 (KR) 2003-0039621

(51) Int. Cl. A61F 9/00

(52) U.S. Cl. 2/12; 2/209.13

(57) ABSTRACT

Disclosed is a hat with a shade panel that is constructed in a simple manner, is convenient to use, is made at a substantially low cost, and has the shade panel placed at a substantially close distance to wearer's eyes to allow him to clearly view surrounding objects. The shade panel is adapted to be mounted on an inner periphery of a rim of the hat that comes in contact with a wearer's forehead, in such a manner as to be foldable by means of folding means.

### BACKGROUND OF THE INVENTION

### CROSS-REFERENCE TO RELATED FOREIGN APPLICATION

This application claims the priority of Republic of Korea Patent Application No. 2003-0039621 filed Dec. 19, 2003, the disclosure of which is incorporated herein by reference in its entirety.

1. Field of the Invention

The present invention relates to a hat with a shade panel, and, more particularly, to a hat with a shade panel that is constructed in a simple manner, is convenient to use, is made at a substantially low cost, and/or has the shade panel placed at a substantially close distance to wearer's eyes to allow him to clearly view surrounding objects.

2. Background of the Related Art

Generally, a hat is usually worn outdoors to give protection from direct sunlight or cold weather. Such a hat is provided with a visor that extends outwardly by a predetermined distance from the body of the hat placed on a wearer's head for the purpose of protecting his face as well as his eyes from the sunlight. In cases where the wearer stays outdoors while spending a long period of time climbing, fishing, swimming, exercising, watching games, etc., he usually wears a hat and a pair of sunglasses such that he can protect his eyes and face from the sunlight and its ultraviolet rays.

Since the hat and the sunglasses are conventionally separate accoutrements, wearers have had problems using both together. For instance, if the wearer wears only the hat, he may keep his sunglasses in a pocket or may hang them around his neck by a cord. However, this way of keeping or wearing sunglasses may be inconvenient for wearers while they are walking and may cause the risk of damage to the sunglasses due to carelessness.

To solve this problem, Korean Utility Model Registration No. 20-199241 discloses a hat with glasses attached thereto, wherein the glasses slide along a guide rail by a predetermined distance. The hat 10, as shown in FIGS. 1 and 2, includes a brim 11 having a guide rail 20 fixed at the bottom surface thereof. The guide rail 20 is fixedly attached to the middle portion of the brim 11 in such a manner as to be directed toward the front side thereof. The guide rail 20 has a guide path 21 formed along a longitudinal direction thereof, which communicates with its opened lower portion. The guide rail 20 has locking protrusions 23a and 23b formed at both sides thereof. A movable adjusting piece 30 is inserted into the guide path 21 such that it can be moved along the guide path 21. To this end, the guide rail 20 is opened at one side thereof.

In more detail, a locking plate 31 of the movable adjusting piece 30 is inserted into the guide rail 20 through the opened side 24 of the guide rail 20. Thereafter, a closing piece 40 is inserted into the opened side 24 of the guide rail 20 to close the guide rail 20 such that the movable adjusting piece 30 is secured within the guide rail 20. The movable adjusting piece 30 includes the locking plate 31 that is inserted into the guide path 21 in such a manner as to be moved along the guide path 21, and a fixing piece 33 that is connected through a connecting part 32 to the locking plate 31, for fixing glasses 50 thereto. The fixing piece 33 is of a generally “ [symbol] ” shape and has a fixing pin 34 that is adapted to connect both side plates thereof to each other. The glasses 50 are pivotally fixed to the fixing pin 34. To this end, the glasses 50 have a locking hook 51 formed at the middle portion of the top end thereof. The locking hook 51 is fitted to the fixing pin 34 through the opened portion thereof, having a certain elasticity. The elasticity of the locking hook 51 enables the fixing pin 34 to be resiliently depressed and allows the glasses 50 to be kept in position, without any motion, in their fixed state when the glasses 50 are folded toward the brim 11 or unfolded for use. The section of the fixing pin 34 against which the locking hook 51 is placed is formed eccentrically to one side thereof. In particular, the fixing pin 34 has a generally diamond shape, and thus, the folded position of the glasses 50 and the wearing position thereof are placed at the edge of the fixing pin 34, such that when the glasses 50 are unfolded or folded, they can be easily pivoted to stop there.

The hat described above suffers from various problems, including the following. First, so as to pivot the glasses 50 on the bottom surface of the brim 11, the hat 10 is provided with a fairly complicated structure that requires the guide rail 20, the movable adjusting piece 30 and the closing piece 40. Second, the glasses 50 are slidably movable along the guide rail 20, but they are separated by a predetermined distance from wearer's eyes. As a result, the wearer cannot focus the glasses 50 on objects, which prevents him from clearly viewing surrounding objects through the glasses 50.

### SUMMARY OF THE INVENTION

Accordingly, the present invention has been made in view of the above-mentioned problems occurring in the prior art.

The object of the present invention is to provide a hat with a shade panel that is constructed in a simple manner, is convenient to use, is made at a substantially low cost, and/or has the shade panel placed at a substantially close distance to wearer's eyes for allowing him to clearly view surrounding objects.

To accomplish the above object, the present invention provides a head covering for covering at least a portion of a wearer's head. The head covering includes a body having a crown which defines a cavity sized and shaped so as to receive at least a portion of a wearer's head. A shade panel is attached to the body such that the shade panel can be folded at least partially into the cavity. More particularly, the shade panel is movable between a first position, in which the shade panel is positioned inside the cavity (e.g., when not in use), and a second position, in which the shade panel is positioned outside of the cavity (e.g., when in use to shade the wearer's eyes and face from the sun).

The first disclosed embodiment of the present invention includes a flexible connecting member as folding or connecting means for allowing the shade panel to be folded into the cavity. The lower end of the connecting member engages the upper end of the shade panel and the upper end of the connecting member engages a rim lining the crown of the hat. Cooperating hook and loop fasteners are affixed to the opposing surfaces of the rim and connecting member, whereby the connecting member is detachably connected to the rim. The shade panel is made of flexible plastic material to facilitate the folding thereof, and is preferably transparent and tinted. In this embodiment, the shade panel is shaped like the lenses of a pair of sunglasses.

The shade panel of the second disclosed embodiment of the invention is similarly shaped like the lenses of a pair of sunglasses, and is made of a material providing flexibility, and preferably transparency. The folding means is a flexible connecting member in this embodiment with its lower end engaging the upper end of the shade panel, and its upper end non-detachably engaging the rim lining the crown of the hat. The upper end of the connecting member is sewn to the inner surface of the rim to form stitching.

The shade panel is larger in the third embodiment, being configured and dimensioned to cover generally the top half of the wearer's face. The shade panel has a generally rectangular shape, which aids in protection of the face proximate to the eyes. This embodiment is well-suited for use by senior citizens and people with light-sensitive skin.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is an exploded, perspective view of a hat with sunglasses attached thereto according to the prior art;

FIG. 2 is a perspective, assembled view of the hat with the sunglasses of FIG. 1;

FIG. 3 is a bottom perspective view of a hat with a shade panel according to a first embodiment of the present invention;

FIG. 4 is a side sectional view of the hat of FIG. 3 with the shade panel unfolded and positioned outside of the hat;

FIG. 5 is a side cross-sectional view of the hat of FIG. 3 with the shade panel folded into the hat;

FIG. 6 is a bottom perspective view of a hat with a shade panel according to a second embodiment of the present invention;

FIG. 7 is a side cross-sectional view of the hat of FIG. 6 with the shade panel unfolded and positioned outside of the hat;

FIG. 8 is a side cross-sectional view of the hat of FIG. 6 with the shade panel folded into the hat;

FIG. 9 is a bottom perspective view of a hat with a shade panel according to a third embodiment of the present invention;

FIG. 10 is a side cross-sectional view of the hat of FIG. 9 with the shade panel unfolded and positioned outside of the hat; and

FIG. 11 is a side cross-sectional view of the hat of FIG. 9 with the shade panel folded into the hat.

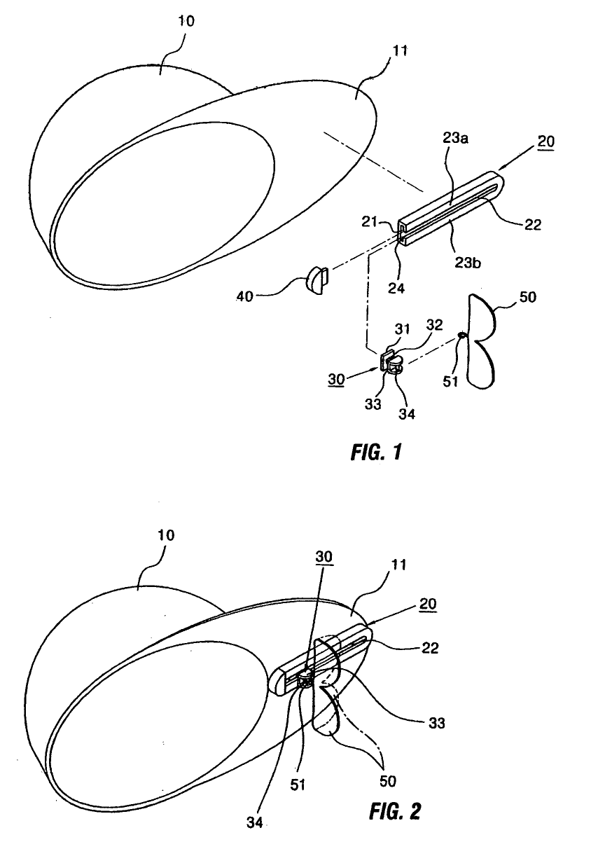

### DETAILED DESCRIPTION OF THE PREFERRED EMBODIMENTS

Reference will now be made in detail to the preferred embodiments of the present invention, examples of which are illustrated in the accompanying drawings.

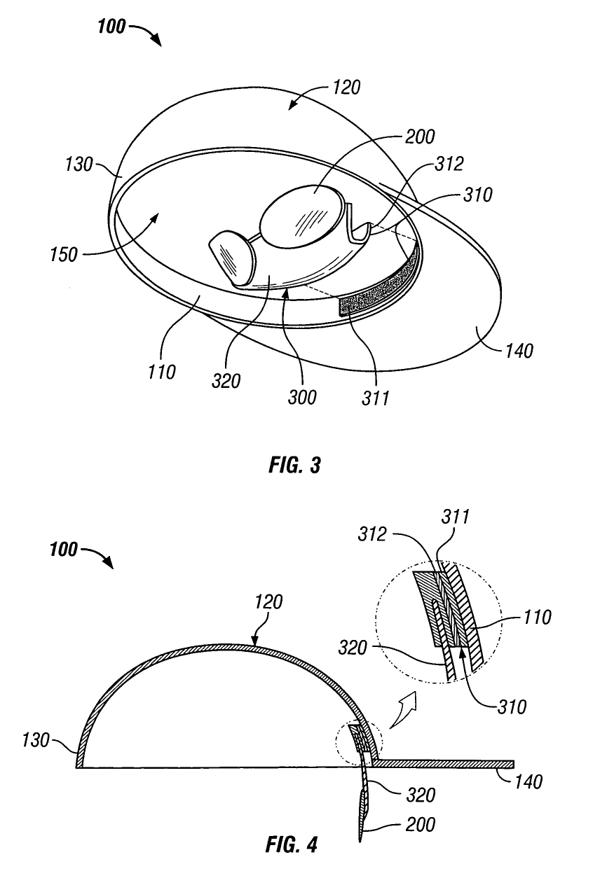

According to the first embodiment of the present invention shown in FIGS. 3-5, a hat 100 has a body 120 which includes a crown 130 and a visor 140 extending from a front part of the crown 130. A rim 110 lines the inner peripheral surface of the crown 130, which defines a cavity 150 for receiving at least a portion of wearer's head. A shade panel 200 is also mounted to the rim 110, preferably where the rim 110 comes in contact with a wearer's forehead, in such a manner as to be foldable by means of folding or connecting means 300. The shade panel 200 is preferably made of flexible plastic material, such as polyethylene, so that when it is folded, it can be disposed inside the cavity 150 defined by the crown 130. The shade panel 200 is also preferably made of a transparent material having color tinting to filter sunlight and protect the wearer's eyes. The shade panel 200 is of a size suitable to cover the wearer's eyes, and thereby protect his eyes from sunlight. In the first and second embodiments, the shade panel 200 is preferably shaped like the lenses of a pair of sunglasses, having left and right ocular portions corresponding to the wearer's eyes.

The folding means 300 includes a flexible connecting member 320. The connecting member 320 is made of a generally flexible material such as stretch fabric, flexible plastic, elastic, or vinyl (polyvinyl chloride), such that it can be unfolded when there is a need to shade the wearer's eyes from sunlight, and then be folded inside the cavity 150 when there is no sunlight. The connecting member 320 is fixed at a lower end thereof to an upper end of the shade panel 200, by means of an adhesive (not shown) like a bond or the like, and is fixed at an upper end thereof to the inner peripheral surface of the rim 110 that comes in contact with the wearer's forehead, by means of fixing means 310. The folding means 300 thereby allows folding of the shade panel 200 into and out of the cavity 150 of the hat 100.

As shown in FIG. 3, the fixing means 310 comprises cooperating hook and loop fastener (Velcro®) members. A first hook and loop fastener member 311 is affixed to the inner peripheral surface of the rim 110 that comes in contact with the wearer's forehead. A second hook and loop fastener member 312 is also affixed to the inner surface of the connecting member 320, opposite to the first hook and loop fastener member 311, in such a manner as to be coupled to the first hook and loop fastener member 311.

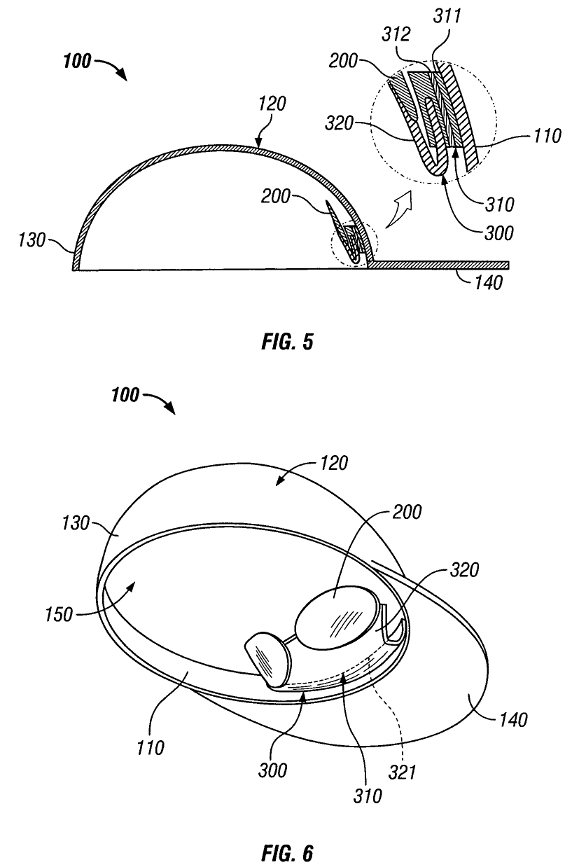

FIG. 4 shows the shade panel 200 unfolded to the outside of the hat 100 (i.e., the cavity 150) to conduct its shade function. FIG. 5 shows the shade panel 200 folded inside the hat 100, so as not to cause inconvenience to the wearer when he wears the hat and is not using the shade panel 200. As described above and shown in FIGS. 3-5, the shade panel 200 is detachably mounted to the rim 110 using the cooperating first and second hook and loop fastener members 311, 312 as the fixing means 310.

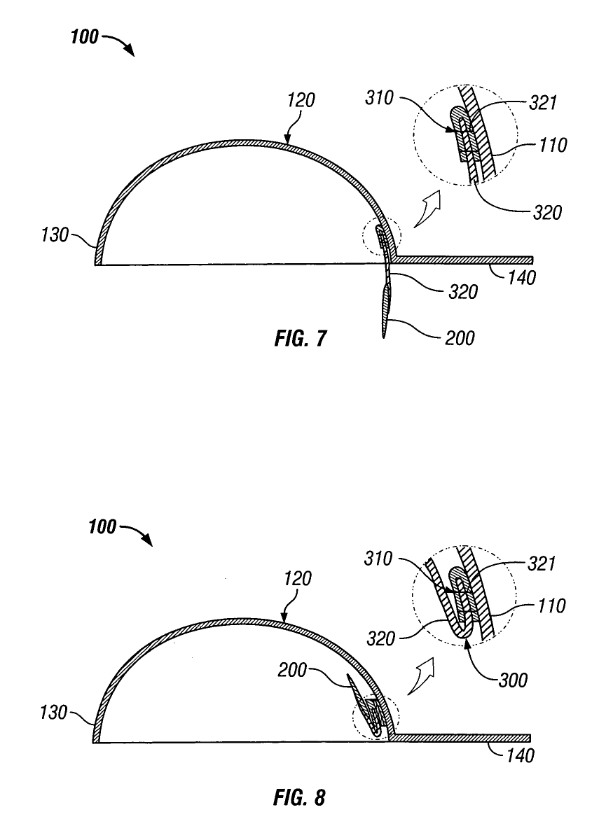

A second embodiment of the present invention is shown in FIGS. 6-8. As in the same manner as the first embodiment, FIG. 7 shows a shade panel 200 unfolded to the outside of a hat 100 to conduct its shade function. FIG. 8 shows the shade panel 200 folded inside the hat 100, so as not to cause inconvenience to the wearer when he wears the hat 100 and is not using the shade panel 200.

Fixing means 310 is formed, as shown in FIGS. 6-8, in such a manner that the upper end of a connecting member 320 is sewn along the inner peripheral surface of a rim 110, thereby forming a stitching 321. The method of sewing the fixing means 310 allows the shade panel 200 to be easily and inexpensively fixed to the rim 110, even though the shade panel 200 is not detachably mounted to the rim 110.

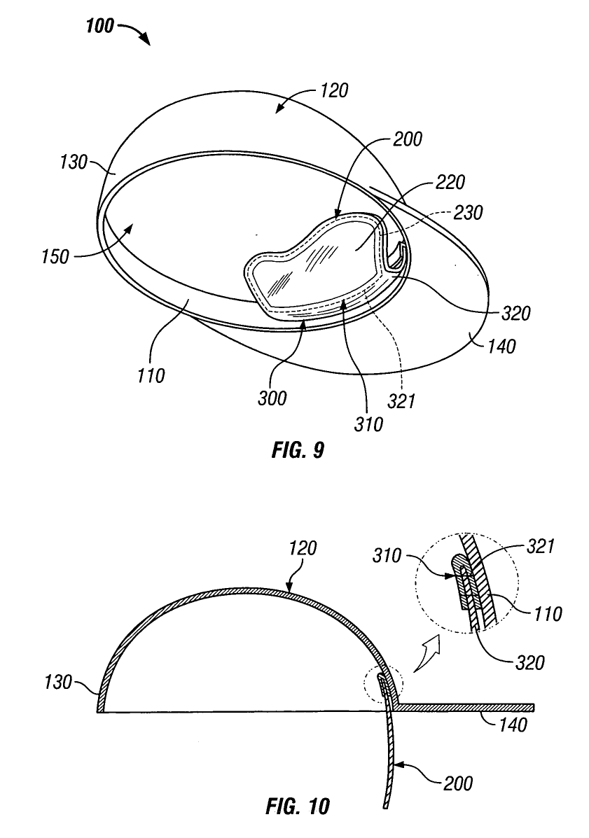

A third embodiment of the present invention is shown in FIGS. 9-11. More particularly, a hat 100 includes a body 120 having a visor 140 and a crown 130 that defines a cavity 150 sized and shaped so as to receive at least a portion of a wearer's head. The hat 100 also includes a shade panel 200 which is configured in a generally rectangular shape and which has an area greater than the sunglasses shape of the embodiments shown in FIGS. 3-8. The shade panel 200 may be of a size sufficient to cover a substantial portion of the wearer's face so as to protect the wearer's face from sunlight. The shade panel 200 comprises a shade member 220 and a lining 230 that covers the outer edge or edges of the shade member 220. The lining 230 may be made of fabric folded over and secured to the outer edges of the shade member 220. The increased surface area of the shade member 220 affords protection of the wearer's eyes while extending coverage to the proximate facial region. Accordingly, this embodiment is well-suited for use by senior citizens and people with light-sensitive skin.

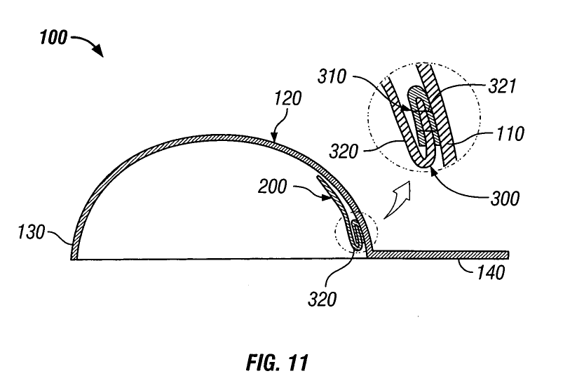

Folding or connecting means 300 of the third embodiment includes a connecting member 320 that is made of a generally flexible material to allow folding of the shade panel 200 into and out of the cavity 150 of the hat 100. Fixing means 310 is formed by sewing the connecting member 320 along the inner peripheral surface of the rim 110 at the upper end thereof, thereby forming a stitching 321. The lower end of the connecting member 320 engages the top of the shade panel 200 where the top part of the lining 230 is affixed to the bottom end of the connecting member 320. FIG. 10 shows the shade panel 200 unfolded to the outside of the hat 100 to conduct its shade function. FIG. 11 shows the shade panel 200 folded inside the hat 100.

The shade member 220 is preferably made of flexible plastic or other suitable material, such as polyethylene. Preferably, the shade member 220 is made of a flexible plastic material that is also resilient, thereby providing the shade panel 200 with a sufficient stiffness to maintain its position when folded into the cavity 150 defined by hat 100. The stiffness afforded by the resiliency of the shade member 220 hence ensures that the shade panel 200 is secured up in the cavity 150 in its folded position without the wearer needing to fix it by use of additional means to prevent the shade panel 200 from falling outside of the cavity 150. The shade member 220 is also preferably made of transparent material having color tinting to filter sunlight and protect the wearer's eyes.

The resilient and flexible material of the shade member 220 also enables the wearer to pull the shade panel 200 out of the cavity with a simple snapping or popping motion when he wishes to shield his eyes and face from the sun. Likewise, the wearer can just as easily snap or pop the shade panel 200 back into the cavity 150 when he no longer needs it. The wearer can therefore easily convert the hat 100 from having the shade panel 200 in the inside (i.e., folded) position to the outside (i.e., unfolded) position and vice versa with a simple fast, direct motion, and can be confident that the shade panel 200 will stay in the position he desires. In other words, the shade panel 200 remains in its inside or outside position without the use of additional supporting or affixing mechanisms (i.e., the shade panel 200 is self-supporting).

As described above, the present invention provides a hat with a shade panel that is constructed in a simple manner, is convenient to use, is made at a substantially low cost, and/or has the shade panel placed at a substantially close distance to wearer's eyes to allow him to clearly view surrounding objects.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

1. A head covering for covering at least a portion of a wearer's head, comprising:
a body including a crown, said crown defining a cavity sized and shaped so as to receive at least a portion of a wearer's head; and
a shade panel attached to said body such that said shade panel can be folded at least partially into said cavity.

2. A head covering according to claim 1, wherein said shade panel is moveable between a first position, in which said shade panel is positioned inside the cavity, and a second position, in which said shade panel is positioned outside of said cavity.

3. A head covering according to claim 2, wherein said crown includes a rim defining an opening of said cavity, and said shade panel being connected to said rim.

4. A head covering according to claim 3, further comprising connecting means for connecting said shade panel to said rim such that said shade panel can be folded into the cavity, said shade panel being attached to said connecting means.

5. A head covering according to claim 4, wherein said connecting means includes affixing means for affixing said connecting means to said rim.

6. A head covering according to claim 5, wherein said affixing means includes cooperating hook and loop fasteners, said fasteners respectively engaging said connecting means and said rim.

7. A head covering according to claim 5, wherein said affixing means includes stitching along the interface of said rim and said connecting means.

8. A head covering according to claim 4, wherein said connecting means includes a connecting member having first and second sides, said first side being affixed to said shade panel, and said second side being affixed to said rim.

9. A head covering according to claim 8, wherein said connecting member is made of a flexible material.

10. A head covering according to claim 1, wherein said shade panel is dimensioned and configured so as to cover a wearer's eyes.

11. A head covering according to claim 10, wherein said shade panel is shaped like lenses of a pair of sunglasses.

12. A head covering according to claim 1, wherein said shade panel is dimensioned and configured so as to cover at least a top part of a wearer's face.

13. A head covering according to claim 1, wherein said shade panel has a generally rectangular shape.

14. A head covering according to claim 2, wherein said shade panel is made of a flexible, resilient material such that it can be moved between its first and second positions in a snapping motion.

15. A head covering according to claim 1, wherein said shade panel is transparent.

16. A head covering according to claim 15, wherein said shade panel is tinted.
